(19) United States
(12) Patent Application Publication
(10) Pub. No.: US 2022/0184026 A1
(43) Pub. Date: Jun. 16, 2022

(54) COMPOSITIONS OF FULVIC ACID AND CANNABINOID AND USES THEREOF

(71) Applicant: Omni Bioceutical Innovations, Inc., Scottsdale, AZ (US)

(72) Inventor: Jane Christensen, Scottsdale, AZ (US)

(21) Appl. No.: 17/686,923

(22) Filed: Mar. 4, 2022

Related U.S. Application Data
(63) Continuation of application No. PCT/US2020/049069, filed on Sep. 2, 2020.
(60) Provisional application No. 62/895,657, filed on Sep. 4, 2019.

(51) Int. Cl. A61K 31/352; A61K 31/05; A61K 31/728; A61K 9/00; A61P 17/02; A61K 45/06

(52) U.S. Cl. CPC A61K 31/352; A61K 31/05; A61K 45/06; A61K 9/0014; A61P 17/02; A61K 31/728

(57) ABSTRACT

Compositions with isolated fulvic acid in combination with a cannabinoid are disclosed herein. Also disclosed are methods of using said compositions for improving cellular regeneration, including the healing, treatment, or prevention of skin disorders.

### CROSS REFERENCE TO RELATED APPLICATIONS

This application is a continuation of PCT Application No. PCT/US2020/049069, filed Sep. 2, 2020, which claims the benefit of U.S. Provisional Application No. 62/895,657, filed Sep. 4, 2019, each of which is hereby incorporated by reference in its entirety.

### FIELD OF THE DISCLOSURE

The present disclosure relates generally to the field of cellular regeneration, including the healing of wounds and treatments and improvements of skin conditions. In particular, the disclosure relates to compositions having isolated bioactive fulvic acid and cannabinoid for use in cellular regeneration.

### BACKGROUND

Humic substances (HS) are ubiquitous in nature and arise from the decay of plant and animal residue in the environment. HS are among the most widely distributed natural products on the surface of the earth, and are the major organic components of soil (humus), lakes, rivers and geological deposits such as peat, leonardite, lignite (brown coal), and organic clays. Humified organic matter (HOM) is relatively stable, but can vary in composition based on its location, deposit type, depth, and age. HOM contains a complex mixture of organic molecules, including bioactive polyelectrolytes (BPs).

BPs include numerous bioactive, naturally occurring, related, but not identical, high molecular-weight polymers. Examples of BPs include, but are not limited to, fractions of HS, such as humic acid (HA), fulvic acid (FA), humin, or ulmic acid (UA). The differences among BPs include a considerable variation in molecular weight and size, the number of functional groups (e.g., carboxyl, phenolic HO), and the extent of polymerization that has taken place. HA and FA have received broad international attention within the scientific community due to their wide range of bioactive characteristics. See, e.g., Drozd J., 1978, Studies of chemical and physiochemical properties of humus compounds of some taxonomic soil units, Rosprawy Naukowe, Zeszyt 13, A R Wroclaw pp. 65. BPs are useful for multiple functions in humans, other animals, and plants.

### SUMMARY

The present disclosure is directed to compositions including an isolated fulvic acid in combination with a cannabinoid, optionally with hyaluronic acid, for use in cell regeneration, including for the treatment of tissue repair and wound healing. Also provided herein are methods of making and using the compositions.

Some embodiments provided herein relate to compositions for treatment of a subject in need of cell regeneration. In some embodiments, the compositions include an isolated fulvic acid having an average molecular weight ranging from 80 to 1200 Da, and a cannabinoid. In some embodiments, the isolated fulvic acid has an average molecular weight ranging from 80 to 350 Da. In some embodiments, the isolated fulvic acid has an average molecular weight ranging from 300 to 320 Da. In some embodiments, the isolated fulvic acid has an average molecular weight of about 308.24 Da. In some embodiments, the isolated fulvic acid has an average molecular weight of 309 Da. In some embodiments, the isolated fulvic acid has an approximate molecular formula of C₁₂H₁₆O₉. In some embodiments, the cannabinoid is cannabidiol. In some embodiments, the composition is formulated as a topical or injectable composition. In some embodiments, the composition further includes hyaluronic acid. In some embodiments, the cannabinoid is present in an amount ranging from about 0.1 to about 100 mg/mL.

In some embodiments, the compositions include an isolated fulvic acid having an average molecular weight ranging from 300 to 320 Da; cannabidiol present in an amount of about 2 mg/mL; and a skin conditioning agent. In some embodiments, the skin conditioning agent is human fibroblast conditioned media. In some embodiments, the compositions further include hyaluronic acid, tocopheryl acetate, phenoxyethanol, polysorbate 20, propylene glycol, 1,2-hexanediol, caprylyl glycol, or combinations thereof. In some embodiments, the composition is formulated as a wound care formulation, a skin conditioner, a face serum, a hair serum, a moisturizer, or a facial booster.

Some embodiments provided herein relate to methods for relieving, improving, or causing regression of a wound or skin condition in a subject in need thereof. In some embodiments, the methods include selecting a subject in need thereof and topically applying a therapeutically effective amount of a topical composition that includes an isolated fulvic acid and a cannabinoid. In some embodiments, the fulvic acid has an average molecular weight of 309 Da and an approximate molecular formula of C₁₂H₁₆O₉. In some embodiments, the cannabinoid is cannabidiol. In some embodiments, the subject suffers from one or more surgical, accidental, or chronic wound or skin condition. In some embodiments, the skin condition is rhytide, non-enzymatic glycosylation of the skin, sun damage, smoking damage, fibrosis of the skin, acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, dyshidrosis, acne mechanica, acne medicamentosa, acne miliaris necrotica (acne varioliformis), acne vulgaris, acne with facial edema (solid facial edema), blepharophyma, erythrotelangiectatic rosacea (erythematotelangiectatic rosacea, vascular rosacea), excoriated acne (acne excoriée des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobate, synovitis-acne-pustulosis-hyperostosis-osteomyelitis syndrome (SAPHO syndrome), steroid rosacea, tar acne, skin cancer (carcinoma and melanoma), tropical acne, plaque psoriasis, guttate psoriasis, inverse psoriasis, pustular psoriasis, erythrodermic psoriasis, nail psoriasis, and psoriatic arthritis.

These features, together with other features herein further explained, will become obvious through a reading of the following description of the drawings and detailed description.

### BRIEF DESCRIPTION OF THE DRAWINGS

The description herein is understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 is a cross-sectional confocal microscopy image captured at 0 micrometers (μm) below the skin surface of a subject treated with a topical formulation that includes an isolated fulvic acid, according to an embodiment of the present disclosure.

FIG. 2 is a cross-sectional confocal microscopy image captured at 20 μm below the skin surface of a subject treated with a topical formulation that includes an isolated fulvic acid, according to an embodiment of the present disclosure.

FIG. 3 is a cross-sectional confocal microscopy image captured at 40 μm below the skin surface of a subject treated with a topical formulation that includes an isolated fulvic acid, according to an embodiment of the present disclosure.

FIG. 4 is a cross-sectional confocal microscopy image captured at 60 μm below the skin surface of a subject treated with a topical formulation that includes an isolated fulvic acid, according to an embodiment of the present disclosure.

FIG. 5 is a cross-sectional confocal microscopy image captured at 80 μm below the skin surface of a subject treated with a topical formulation that includes an isolated fulvic acid, according to an embodiment of the present disclosure.

FIG. 6 is a cross-sectional confocal microscopy image captured at 100 μm below the skin surface of a subject treated with a topical formulation that includes an isolated fulvic acid, according to an embodiment of the present disclosure.

FIG. 7 is a cross-sectional confocal microscopy image captured at 120 μm below the skin surface of a subject treated with a topical formulation that includes an isolated fulvic acid, according to an embodiment of the present disclosure.

FIG. 8 is a cross-sectional confocal microscopy image captured at 140 μm below the skin surface of a subject treated with a topical formulation that includes an isolated fulvic acid, according to an embodiment of the present disclosure.

FIG. 9 is a cross-sectional confocal microscopy image captured at 30 μm below the skin surface of a subject treated with a topical formulation of the present disclosure in the absence of an isolated fulvic acid.

FIG. 10 is a cross-sectional confocal microscopy image captured at 60 μm below the skin surface of a subject treated with a topical formulation of the present disclosure in the absence of an isolated fulvic acid.

FIG. 11 is a cross-sectional confocal microscopy image captured at 100 μm below the skin surface of a subject treated with a topical formulation of the present disclosure in the absence of an isolated fulvic acid.

FIG. 12A is a cross-sectional confocal microscopy image captured at 10 μm below the skin surface of a subject treated with commercial topical formulation, Serum 2, in the absence of an isolated fulvic acid.

FIG. 12B is a cross-sectional confocal microscopy image captured at 50 μm below the skin surface of a subject treated with commercial topical formulation, Serum 2, in the absence of an isolated fulvic acid.

FIG. 13A is a cross-sectional confocal microscopy image captured at 1 μm below the skin surface of a subject treated with commercial topical formulation, Serum 3, in the absence of an isolated fulvic acid.

FIG. 13B is a cross-sectional confocal microscopy image captured at 20 μm below the skin surface of a subject treated with commercial topical formulation, Serum 3, in the absence of an isolated fulvic acid.

FIG. 14A is a cross-sectional confocal microscopy image captured at 0 μm below the skin surface of a subject treated with commercial topical formulation, Serum 4, in the absence of an isolated fulvic acid.

FIG. 14B is a cross-sectional confocal microscopy image captured at 80 μm below the skin surface of a subject treated with commercial topical formulation, Serum 4, in the absence of an isolated fulvic acid.

FIG. 15 is a schematic representation showing skin layers, and demonstration the depth of penetration of various formulations, including: 1 (Serum 1, lacking M-007, penetrating to a depth of 10 μm); 2 (Serum 2, commercially available serum penetrating to a depth of 50 μm); 3 (Serum 3, commercially available serum penetrating to a depth of 20 μm); 4 (Serum 4, commercially available serum penetrating to a depth of 80 μm); and 5 (present compositions including M-007, penetrating to a depth of at least 140 μm).

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Embodiments provided herein relate to compositions for use in cellular regeneration that include a fulvic acid and a cannabinoid. In some embodiments, the compositions further include hyaluronic acid. Also provided are methods of making and using the composition.

1. Compositions

Some embodiments provided herein relate to a composition comprising a fulvic acid and a cannabinoid, optionally with hyaluronic acid. As used herein, the term “composition” or “formulation” has its ordinary meaning in light of the specification, and refers to a combination of elements, components, or compositions presented together for a given purpose.

As used herein, the term “fulvic acid” or “fulvate” has its ordinary meaning when understood in light of the specification, and refers to a fraction of humic substances that is soluble in water under all pH conditions. It is also soluble in methyl ethyl ketone, methyl alcohol, and acids. It generally has a yellow (fulvus) to yellow-brown color. Fulvate includes a mixture or collection of different acids containing carboxyl and phenolate groups. The structure of fulvate contains both aromatic and aliphatic structures that are extensively substituted with oxygen-containing functional groups. A proposed fulvate structure has been previously described (see Buffle J., Greter F. L., Haerdi W., 1977, Measurements of Complexation Properties of Humic and Fulvic Acids in Natural Water, With Lead & Copper Ion-Selective Electrodes. Anal. Chem. 49: 216-222; see also US Patent Application No. 2015/0216839; each of which are incorporated herein by reference in their entireties). As used herein, the term “fulvate” encompasses the esters, salts or ion complexes of fulvic acid. Fulvic acid isolated from humic matter may include a range of specific fulvic acid compounds, including up to 27 distinct fulvic acid fractions.

In some embodiments, the compositions provided herein include an isolated fulvic acid and a cannabinoid. In some embodiments, the isolated fulvic acid is a specific fulvic acid or family of fulvic acids having an average molecular weight ranging from about 80 Da to about 350 Da. In some embodiments, the isolated fulvic acid is a fulvic acid having an average molecular weight ranging from about 300 Da to 320 Da. In some embodiments, the isolated fulvic acid is a fulvic acid having an average molecular weight of about 309 Da. In some embodiments, the isolated fulvic acid is a fulvic acid having an average molecular weight of about 308.24 Da. In any of the embodiments described herein, the compositions further include hyaluronic acid. In any of the embodiments described herein, the average molecular weight is measured by vapor pressure osmometry. As described herein, “vapor pressure osmometry” has its ordinary meaning as understood in light of the specification, and refers to a nonspectroscopical technique for measuring the number average molecular weight of a polymer. In some embodiments, the isolated fulvic acid has a molecular formula of C₁₂H₁₆O₉. A fulvic acid compound having an average molecule weight ranging from about 300 Da to about 320 Da, and having a molecular formula of C₁₂H₁₆O₉ is referred to herein as M-007.

In some embodiments, the isolated fulvic acid is extracted and isolated using an isolation method as set forth in U.S. Pat. No. 9,820,953, including the steps of providing an aqueous slurry having humified organic matter (HOM); applying the aqueous slurry to high pressure column fractionation to obtain fractionated samples; applying the fractionated samples to molecular sieving; and isolating a fulvic acid. In some embodiments, the isolated fulvic acid is present in the composition in an amount ranging from about 0.0001% w/v to about 99% w/v, such as 0.0001, 0.0005, 0.001, 0.005, 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99% w/v, or in an amount within a range defined by any two of the aforementioned values.

The term “isolated” is meant material that is substantially or essentially free from components that normally accompany it in its native state. For example, an “isolated fulvic acid,” as used herein, includes a fulvic acid that has been purified from its naturally-occurring state, e.g., a fulvic acid that has been removed from the humified organic matter in which it naturally resides.

In some embodiments, the compositions include a cannabinoid. As used herein, the term “cannabinoid” has its ordinary meaning as understood in light of the specification, and refers to a compound within the class that acts upon cannabinoid receptors (such as on the endocannabinoid system) in cells. A cannabinoid can include a synthetic cannabinoid, a phytocannabinoid, or an endocannabinoid. In some embodiments, a cannabinoid includes cannabidiols (including cannabidiol, cannabidiolic acid, cannabidiol monomethylether, cannabidiorcol, cannabidivarin, or cannabidivarinic acid), tetrahydrocannabinols (including delta-9-tetrahydrocannabinol, delta-9-cis-tetrahydrocannabinol, delta-9-tetrahydrocannabinol-C4, delta-9-tetrahydrocannabinolic acid A, delta-9-tetrahydrocannabinolic acid B, delta-9-tetrahydrocannabinolic acid C4, delta-9-tetrahydrocannabiorcol, delta-9-tetrahydrocannabiocolic acid, delta-9-tetrahydrocannabivarin, delta-9-tetrahydrocannabivarinic acid, tryhydroxy-delta-9-tetrahydrocannabinol, delta-8-tetrahydrocannabinol, or delta-8-tetrahydrocannabinolic acid), cannabichromenes (including cannabichromene, cannabichromenenic acid, cannabichromevarin, cannabichromenvarinic acid), cannabigerols (including cannabigerol, cannabigerol monomethylether, cannabigerolic acid, cannabigerolic acid monomethylether, cannabinerolic acid, cannabigerovarin, or cannabigerovarinic acid), cannabielsoins (including cannabielsoin, cannabielsoic acid A, or cannabielsoic acid B), cannabinols or cannabinodiols (includiong cannabinodiol, cannabinodivarin, cannabinol, cannabinol methylether, cannabinol-C2, cannabinol-C4, cannabinolic acid, cannabiorcool, or cannabivarin), cannabitriols (including cannabitriol, cannabitriolvarin, 10-ethoxy-9-hydroxy-delta-6a-tetrahydrocannabinol, or 8,9-dihydroxy-delta-6a-tetrayhydrocannabinol), cannabicyclols, 10-oxo-delta-6a-tetrahydrocannabinol, cannabichromanon, cannabifuran, cannabiglendol, cannabiripsol, cannbicitran, dehydrocannabifuran, cannabicitran, or 3,4,5,6-tetrahydro-7-hydroxy-alpha-alpha-trimethyl-9-n-propyl-2,6-methano-2H-1-benzoxicin-5-methanol, or any analogue or derivative thereof In some embodiments, the cannabinoid is cannabidiol. In some embodiments, the cannabinoid is present in the composition in an amount ranging from about 0.0001% w/v to about 99% w/v, such as 0.0001, 0.0005, 0.001, 0.005, 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99% w/v, or in an amount within a range defined by any two of the aforementioned values.

In some embodiments, the compositions include hyaluronic acid. As used herein, the term “hyaluronic acid” has its ordinary meaning as understood in light of the specification, and refers to a polymer of disaccharides, themselves composed of D-glucuronic acid and N-acetyl-D-glucosamine, linked via alternating β-(1→4) and β-(1→3) glycosidic bonds. Hyaluronic acid is also referred to herein as hyaluronan or hyaluronate. In some embodiments, hyaluronic acid is present in the composition in an amount ranging from about 0.0001% w/v to about 99% w/v, such as 0.0001, 0.0005, 0.001, 0.005, 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99% w/v, or in an amount within a range defined by any two of the aforementioned values.

Accordingly, some embodiments provided herein relate to compositions that include an isolated fulvic acid having an average molecular weight ranging from about 300 Da to about 320 Da and having a molecular formula of C₁₂H₁₆O₉ (M-007) in combination with cannabidiol, optionally in combination with hyaluronic acid. In some embodiments, the ratio of M-007 to cannabidiol is a ratio of 1:100, 1:90, 1:80, 1:70, 1:60, 1:50, 1:40, 1:30, 1:20, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 20:1, 30:1, 40:1, 50:1, 60:1, 70:1, 80:1, 90:1, or 100:1, or at a ratio within a range defined by any two of the aforementioned ratios.

The compositions provided herein may further included suitable pharmaceutically acceptable carriers, stabilizers, diluents, buffers, or components for application, storage, bioavailability, solubility, or other component parts that improve the efficacy, aesthetics, or other properties of the compositions.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as topical, oral, or intravenous application, and administered in the form of a conventional pharmaceutical preparation, such as solid such as tablets, granules, powders, capsules, caplets, and liquid such as solution, spray, emulsion, foam, suspension, cream, lotion, ointment, salve, gel, and the like. Often the physiologically acceptable carrier is an aqueous pH buffered solution such as phosphate buffer or citrate buffer. The physiologically acceptable carrier may also include, for example, one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counter ions such as sodium, and nonionic surfactants such as Tween™, polyethylene glycol (PEG), and Pluronics™. Auxiliary, stabilizer, emulsifier, lubricant, binder, pH adjustor controller, isotonic agent, and other conventional additives may also be added to the carriers.

The pharmaceutically acceptable or appropriate carrier may include other compounds known to be beneficial to an impaired situation of the skin, (e.g., antioxidants, such as Vitamin C, Vitamin E, Selenium or Zinc), or a food composition. The food composition can be, but is not limited to, milk, yogurt, curd, cheese, fermented milks, milk based fermented products, ice-creams, fermented cereal based products, milk based powders, infant formulae, tablets, liquid bacterial suspensions, dried oral supplement, or wet oral supplement.

As described herein, the composition including an isolated fulvic acid and a cannabinoid can be a topical formulation. The topical formulation can further include, for example, a pharmaceutical vehicle that does not interfere with the function and viability of the isolated fulvic acid or the cannabinoid. The “pharmaceutical vehicle” as described herein refers to an inert substance with which a medication is mixed to facilitate measurement and administration of the topical formulation.

In some embodiments, the active ingredients and mixtures of active ingredients can be used, for example, in topical formulations including a pharmaceutically acceptable carrier prepared for storage and subsequent administration. As used herein, “topical” refers to the administration or application of a formulation to the skin or various body orifices. Some embodiments include use of the fulvic acid and cannabinoid described herein in combination with a pharmaceutically acceptable carrier or diluent. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives and stabilizers can be provided in the topical formulation. Preservatives can be used to keep the nutrients for the skin cells from breaking down. As used herein, the terms “carrier or diluent” may be a solid carrier or diluent for solid formulations, a liquid carrier or diluent for liquid formulations, or mixtures thereof. Solid carriers/diluents include, but are not limited to, a gum, a starch (e.g., cornstarch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. For liquid formulations, such as for topical or parenteral formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Additional components in the compositions can include, for example, water, human fibroblast conditioned media, propylene glycol, lonicera caprifolium (honeysuckle) flower extract, lonicera japonica (honeysuckle) flower extract, 1,2-hexanediol, or caprylyl glycol, or combinations thereof.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

Topical formulations including an isolated fulvic acid and a cannabinoid can be formulated and used as a solution, spray, emulsion, foam, suspension, cream, lotion, ointment, salve, or gel for topical application. Suitable ingredients in the topical formulation can include a for example, water, saline, dextrose, mannitol, lactose, lecithin, albumin, or sodium glutamate, and the like. If desired, absorption enhancing preparations (for example, liposomes), can be utilized.

As used herein, the term “injectable composition” refers to a formulation that is prepared for administration by injection. These injections may be administered by such routes as intravenous, subcutaneous, intradermal, intramuscular, intraarticular, or intrathecal.

In some embodiments, the pharmaceutical vehicle is soybean, grapefruit or almond oils, or synthetic fatty acid esters, such as ethyl oleate or triglycerides, or liposomes.

Coconut oil, olive oil, sesame oil, peanut oil, and soya can be used as suspension agents or lubricants in the topical formulation.

The topical formulation including an isolated fulvic acid and a cannabinoid can further include, for example, one or more solvents, at least one botanical, and/or at least one emollient.

2. Methods of Treating a Disorder

Embodiments provided herein relate to methods of administering the compositions provided herein for improving cellular regeneration on a subject. In some embodiments, the methods include administering a composition including M-007 and cannabidiol to a subject. In some embodiments, administering the compositions to a subject treats a disorder or a wound of a subject, such as a skin disorder or a skin wound. In some embodiments, administering the compositions to the subject prevents development or start of a disorder or wound, such as a skin disorder or skin wound.

As used herein, the term “treatment” refers to an intervention made in response to a disease, disorder or physiological condition manifested by a subject, particularly a subject suffering from one or more wound or skin disorder. The aim of treatment may include, but is not limited to, one or more of the alleviation or prevention of symptoms, slowing or stopping the progression or worsening of a wound, disease, disorder, or condition and the remission of the wound, disease, disorder, or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments, treatments reduce, alleviate, or eradicate the symptom(s) of the disease(s).

As used herein, the term “prevention” refers to any activity that reduces the burden of the individual later expressing disease symptoms. This can take place at primary, secondary, and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

The term “half maximal effective concentration” or “EC₅₀” refers to the concentration of an antibody or other agent described herein at which it induces a response halfway between the baseline and maximum after some specified exposure time; the EC₅₀ of a graded dose response curve therefore represents the concentration of a compound at which 50% of its maximal effect is observed. In certain embodiments, the EC₅₀ of an agent provided herein is indicated in relation to activity related to symptoms or pathology of skin disorders. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC₅₀” and the Hill slope, or it can be determined from the data directly, using routine knowledge in the art. In some embodiments, the EC₅₀ of an antibody or other agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, or 100 nM. Preferably, biotherapeutic compositions will have an EC₅₀ value of about 1 nM or less.

The term “modulating” includes “increasing” or “stimulating,” as well as “decreasing” or “reducing,” typically in a statistically significant or a physiologically significant amount as compared to a control. An “increased” or “enhanced” amount is typically a “statistically significant” amount, and may include an increase that is 1.1, 1.2, 2, 3, 4, 5, 6, 7, 8, 9, 10, 15, 20, 30, or more times (e.g., 500, 1000 times) (including all integers and decimal points in between and above 1, e.g., 1.5, 1.6, 1.7, 1.8, etc.) the amount produced by no composition (the absence of an agent or compound) or a control composition. A “decreased” or reduced amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18% , 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between. As one non-limiting example, a control in comparing canonical and non-canonical activities could include the activity (e.g., antagonist activity) or of a formulation composition, such as, for example, a fulvic acid and a cannabinoid, towards a skin disorder relative to no treatment, a placebo treatment, or a control treatment. Other examples of “statistically significant” amounts are described herein.

A “subject,” as used herein, includes any animal that exhibits a symptom, or is at risk for exhibiting a symptom, of one or more disorder such as rhytide, non-enzymatic glycosylation of the skin, sun damage, smoking damage, fibrosis of the skin, acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, dyshidrosis, acne mechanica, acne medicamentosa, acne miliaris necrotica (acne varioliformis), acne vulgaris, acne with facial edema (solid facial edema), blepharophyma, erythrotelangiectatic rosacea (erythematotelangiectatic rosacea, vascular rosacea), excoriated acne (acne excoriée des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobate, synovitis-acne-pustulosis-hyperostosis-osteomyelitis syndrome (SAPHO syndrome), steroid rosacea, tar acne, skin cancer (carcinoma and melanoma), tropical acne, psoriasis, including plaque psoriasis, guttate psoriasis, inverse psoriasis, pustular psoriasis, erythrodermic psoriasis, nail psoriasis, psoriatic arthritis, or combinations thereof, among others described herein and known in the art. Also included are subjects for whom it is desirable to profile presence and/or levels of disorder-associated markers, for diagnostic or other purposes. In certain aspects, a subject includes any animal having a wound or skin disorder, as described herein and known in the art. Suitable subjects (patients) include laboratory animals (such as mouse, rat, rabbit, or guinea pig), farm animals, and domestic animals or pets (such as a cat or dog). Non-human primates and, preferably, human patients, are included.

In some embodiments, the compositions provided herein improve wound regeneration, improve wound healing, improve hair growth, improve sun protection, improve skin appearance, or improve a disorder associated with skin.

The formulations provided herein having M-007 and cannabidiol may be formulated for topical application. In some embodiments, topical application of the formulations having M-007 and cannabidiol regulates over 150 genes in the skin, including, genes related to wound healing and growth factors, genes connected to skin hydration, genes related to skin barrier integrity, genes related to skin pigmentation, genes related to inflammation and immune response, genes involved in ECM breakdown and barrier integrity, genes relevant to acne, genes connected to anti-aging functions, genes linked to antioxidant and stress responses, genes related to regulating circadian rhythm, and/or genes involved in epidermal barrier formation.

In some embodiments, the methods include administering the compositions provided herein to a subject to inhibit, prevent, or treat a disease, disorder, ailment, and or damage of skin, hair, and/or nails. In some embodiments, the subject can have an oral disease of the mouth. In some embodiments, the subject suffers from an ocular disease of the ear. In some embodiments, the subject suffers from inflammation. In some embodiments, the subject can have a wound.

Besides providing a structural barrier, the skin contains several immune cells that can be activated by invading pathogens or skin damage. One of the most important immune cells involved in wound healing is the macrophage, which exhibits different immunological functions in the skin, including phagocytosis and antigen-presentation. Furthermore, they can produce many cytokines and chemokines to orchestrate the wound healing process throughout the different phases.

“Skin damage” as described herein, can refer to damage to the skin that can be caused by aging, sun damage, cancer, skin disorder or skin diseases that can cause irritation of the skin. Without being limiting, the “skin diseases” and/or “skin disorders” can include rhytide, non-enzymatic glycosylation of the skin, sun damage, smoking damage, fibrosis of the skin, acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, acne mechanica, acne medicamentosa, acne miliaris necrotica (acne varioliformis), acne vulgaris, acne with facial edema (solid facial edema), blepharophyma, erythrotelangiectatic rosacea (erythematotelangiectatic rosacea, vascular rosacea), excoriated acne (acne excoriée des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobate, synovitis-acne-pustulosis-hyperostosis-osteomyelitis syndrome (SAPHO syndrome), steroid rosacea, tar acne, skin cancer, tropical acne, psoriasis, including plaque psoriasis, guttate psoriasis, inverse psoriasis, pustular psoriasis, erythrodermic psoriasis, nail psoriasis, psoriatic arthritis, and combinations and/or variations thereof. In some embodiments described herein, a method of treating a subject in need is provided. The subject can have a disease affecting the skin as provided described herein.

“Hair and scalp disorders” are diseases that affect the hair and scalp and are also described herein. Diseases that affect hair and scalp can include but are not limited to alopecia, androgenic alopecia, hirsutism, hair shaft disorders, inflammation, acromegaly, eczema, psoriasis, impetigo, atopic dermatitis, darier disease, and folliculitis. Common causes for scalp disorders can include but are not limited to acromegaly, atopic dermatitis, darier disease, eczema, fragile X syndrome, impetigo, pachydermoperiostosis, psoriasis, and Rosenthal-Kloepfer syndrome. In some embodiments described herein, a method of treating a subject in need is provided. The subject can have a disease affecting the skin and scalp. In some embodiments the subject suffers from alopecia, androgenic alopecia, hirsutism, hair shaft disorders, inflammation, acromegaly, eczema, psoriasis, impetigo, atopic dermatitis, darier disease, and/or folliculitis. In some embodiments, the subject suffers from acromegaly, atopic dermatitis, darier disease, eczema, fragile X syndrome, impetigo, pachydermoperiostosis, psoriasis, and/or Rosenthal-Kloepfer syndrome. In some embodiments, the treating includes administering a formulation to the subject in need. In some embodiments, the formulation is within a hair cream, a hair gel, a scalp lotion, a shampoo, conditioner, hair spray, or a hair mousse.

“Nail diseases” are disorders or diseases that affect the nail, nail bed, or cuticle region and are also described herein. Diseases that affect the nail and surrounding skin area such as the cuticle can lead to infection or inflammation that could require medical assistance. Diseases that infect the nail, nail bed, and/or cuticle can include but is not limited to onychia, onchyocryptosis, onychodystophy, onychogryposis, onycholysis, onychomadesis, onychomycosis, tinea unguium, onychophosis, onychoptosis, onchorrhexis, paronychia, Koilonychia, subungual hematoma, onychomatricoma, nail pemphigus, erythronychia, and melanonychia. In some embodiments described herein, a method of treating a subject in need is provided. The subject can have a disease affecting the nails, nail bed, and/or cuticles. In some embodiments, the subject suffers from onychia, onchyocryptosis, onychodystophy, onychogryposis, onycholysis, onychomadesis, onychomycosis, tinea unguium, onychophosis, onychoptosis, onchorrhexis, paronychia, Koilonychia, subungual hematoma, onychomatricoma, nail pemphigus, erythronychia, and/or melanonychia. In some embodiments, the treating includes administering a formulation to the subject in need. In some embodiments, the formulation is within a skin cream, a lotion, a cuticle cream, or a nail polish.

“Oral health” as described herein, refers to the health of the teeth and the surrounding tissues such as the gums. Poor oral health can arise from poor oral hygiene, tooth decay, gum disease, diabetes, pregnancy, cancer, HPV, oral cancer (squamous cell carcinoma, verrucous carcinoma, minor salivary gland carcinomas, lymphomas), benign oral cavity, and oropharyngeal tumors (eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors), leukoplakia and erythroplakia, and tongue cancer. Cancer in the mouth can occur on and around the tongue, the gums, the roof of the mouth, and in the insides of the cheeks and lips. In some embodiments, a treatment is provided for maintenance of oral health for a subject in need. In some embodiments, the subject has poor oral hygiene, tooth decay, gum disease, diabetes, cancer, HPV, oral cancer (squamous cell carcinoma, verrucous carcinoma, minor salivary gland carcinomas, lymphomas), benign oral cavity, and oropharyngeal tumors (eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors), leukoplakia and erythroplakia, or tongue cancer. In some embodiments, the subject is pregnant. In some embodiments, the treatment includes administering to the subject in need. In some embodiments, the formulation is in the form of a gargle or a rinse. In some embodiments, the formulation is in a gel, and the gel is administered in a teeth tray. In some embodiments, the formulation is a toothpaste, a prophylactic paste, a tooth polish, a dental solution, an oral spray, an oral rinse, a mouth wash, dental floss, chewing gum, a lozenge, or a tablet.

“Inflammation” as described herein, refers to a biological response of a body tissue to harmful stimuli. The harmful stimuli can include but is not limited to pathogens, bacteria, viruses, fungi, damaged cells, and other irritants that are known to those skilled in the art. Inflammation can be a protective immune response that can involve, for example, immune cells, white blood cells, blood vessels, molecular mediators, and other small molecules. Signs of inflammation can include but is not limited to pain, heat, swelling, and/or loss of function. Inflammation can be acute or chronic. In some embodiments described herein, a formation is provided for the treatment of inflammation. In some embodiments, the subject suffers from inflammation. In some embodiments, the inflammation is on the skin, scalp, nasal passages, mouth, nail area such as the cuticles, eyes, vaginal area or the perineal area.

“Auditory health” can refer to the health of the ear, inner ear, outer ear and surrounding areas. Auditory care can be required when a subject has inflammation in the ear, the ear canal and the surrounding tissues. Common irritants such as bacteria, viruses, mucus, and other skin conditions can lead to the inflammation of the ear. External irritations can also occur that can cause inflammation of the ear. For example, when water gets trapped in the ear canal, bacteria can spread which can then cause inflammation and pain. Inner ear inflammation can also occur following a viral infection such as flu or upper respiratory infection. The virus can then cause swelling of the balance organs leading to dizziness with or without pain during inner ear inflammation.

As used herein, the term “high pressure column fractionation” refers to the use of at least one column under high pressure to separate unreacted components, reaction by-products, and/or low molecular weight excipients, thereby fractionating the slurry into various fractions based on size and properties.

As used herein, the term “molecular sieving” refers to passage of a sample through a one or more porous material having pore diameters of less than 50, 40, 30, 20, 10, 9, 8, 7, 6, 5, 4, 3, 2, 1, or 0.1 nm. The samples may be passed through such molecular sieves in tandem in order to obtain fractions of various size and properties from the aqueous slurry. In some embodiments, high-pressure column fractionation can be used alone or in combination with molecular sieving to obtain a desired fulvic acid that is combined with a cannabinoid for use in the treatment of skin diseases, disorders, or wounds.

In some embodiments, the methods include administering to the subject a therapeutically effective amount of a composition, wherein the composition includes an isolated fulvic acid and a cannabinoid. In some embodiments, the isolated fulvic acid is M-007 and the cannabinoid is cannabidiol. In some embodiments, the composition further includes pharmaceutically acceptable carriers, stabilizers, diluents, buffers, or components for application, storage, bioavailability, solubility, or other component parts, or combinations thereof, that improve the efficacy, aesthetics, or other properties of the compositions.

As used herein, the term “therapeutically effective amount” means on amount of a composition comprising an isolated fulvic acid and cannabinoid which is capable of alleviating, relieving, preventing, ameliorating, or eliminating a disease state for which administration of the composition is indicated. In some embodiments, a therapeutically effective amount includes administration of an amount of isolated fulvic acid ranging from about 0.01 mg/kg to about 200 mg/kg, such as 0.01, 0.05, 0.1, 0.5, 1, 2, 3, 4, 5, 10, 20, 30, 40, 50 60, 70, 80, 90, 100, 110, 120, 130, 140, 150, 160, 170, 180, 190, or 200 mg/kg or an amount within a range defined by any two of the aforementioned amounts. In some embodiments, the composition is formulated having an amount of isolated fulvic acid ranging from about 0.001 to about 50 μg/mL, such as 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 25, 30, 35, 40, 45, or 50 μg/mL, or an amount within a range defined by any two of the aforementioned values. In some embodiments, a therapeutically effective amount includes administration of an amount of cannabinoid ranging from about 0.01 mg/kg to about 200 mg/kg, such as 0.01, 0.05, 0.1, 0.5, 1, 2, 3, 4, 5, 10, 20, 30, 40, 50 60, 70, 80, 90, 100, 110, 120, 130, 140, 150, 160, 170, 180, 190, or 200 mg/kg or an amount within a range defined by any two of the aforementioned amounts. In some embodiments, the composition is formulated having an amount of cannabinoid ranging from about 0.001 to about 50 μg/mL, such as 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 25, 30, 35, 40, 45, or 50 μg/mL, or an amount within a range defined by any two of the aforementioned values.

In some embodiments, the compositions further include a therapeutically effective amount of hyaluronic acid. In some embodiments, a therapeutically effective amount includes hyaluronic acid in an amount ranging from about 0.01 mg/kg to about 200 mg/kg, such as 0.01, 0.05, 0.1, 0.5, 1, 2, 3, 4, 5, 10, 20, 30, 40, 50 60, 70, 80, 90, 100, 110, 120, 130, 140, 150, 160, 170, 180, 190, or 200 mg/kg or an amount within a range defined by any two of the aforementioned amounts. In some embodiments, the composition is formulated having an amount of hyaluronic acid ranging from about 0.001 to about 50 μg/mL, such as 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 25, 30, 35, 40, 45, or 50 μg/mL, or an amount within a range defined by any two of the aforementioned values.

In some embodiments, the methods include selecting a subject who is in need of receiving the compositions provided herein. Selecting a subject in need includes identifying a subject having or suspected of having one or more of the disorders, diseases, or wounds as described herein, and electing to administer treatment to said subject. In some embodiments, a subject is selected for prophylactic measures, to prevent the onset of a disorder or disease.

In some embodiments, the treatment alleviates or relieves a skin disorder. As described herein, the term “alleviate” or “relieve” refers to an improvement in the skin disorder, disease, or wound. In some embodiments, an improvement includes an enhancement of the visual and/or sensory aspects of the superficial layers of the epidermis, as a result in the healing of the skin disorder, disease, or wound.

In some embodiments, the subject suffers from a skin disorder. In some embodiments, the subject suffers from pain. The pain can come from headaches, stomachaches, cancer, autoimmune disease, or a genetic disorder. In some embodiments, the pain is derived from a skin irritation, skin inflammation, systemic issues such as pain arising from joints, muscles, organs, and other sites of inflammation.

In some embodiments, the subject suffers from inflammation. In some embodiments, the subject suffers from inflammation. In some embodiments, the inflammation is on the skin, scalp, nasal passages, mouth, nail area such as the cuticles, eyes, vaginal area or the perineal area.

In some embodiments, the subject has a skin disorder. In some embodiments, the skin disorder is acne, alopecia, alopecia areata, alopecia totalis, angioma, athletes foot, Bowen's disease, carbuncles, candidiasis, cellulitis, dermatitis, eczema, atopic dermatitis, contact dermatitis, seborrheoeic dermatitis, stasis dermatitis, dermatofibroma, echtima, epidermolysis bullosa, erythrasma, folliculitis, Hidradentitis suppurativa, hives, hyperhidrosis, ichthyosis, impetigo, Kaposi's sarcoma, keloid, keratoacanthoma, keratosis, keratosis pilaris, keratosis follicularis, lichen planus, melanoma, melisma, miliaria, pedifulosis, pemphigus, pityriasis rosea, pityriasis rubra pilaris, psoriasis, Raynaud's disease, ringworm, rosacea, scabies, scleroderma, sebaceous cyst, skin cancer, skin tags or shingles. In some embodiments, the subject is suffering effects of skin aging. In some embodiments, the effects of skin aging may include, for example, wrinkling of skin, sunspots, sagging, and loss of skin collagen. In some embodiments, the subject suffers from a skin disease or disorder such as acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, acne mechanica, acne medicamentosa, acne miliaris necrotica (acne varioliformis), acne vulgaris, acne with facial edema (solid facial edema), blepharophyma, erythrotelangiectatic rosacea (erythematotelangiectatic rosacea, vascular rosacea), excoriated acne (acne excoriée des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobate, synovitis-acne-pustulosis-hyperostosis-osteomyelitis syndrome (SAPHO syndrome), steroid rosacea, tar acne, skin cancer, tropical acne, psoriasis, including plaque psoriasis, guttate psoriasis, inverse psoriasis, pustular psoriasis, erythrodermic psoriasis, nail psoriasis, psoriatic arthritis, or combinations and variations thereof.

In some embodiments, the subject in need suffers from a skin disorder or a subject having a skin disorder is selected to receive a therapeutic. In some embodiments, the therapeutic is retin A, hydroquinone, retinol, or an antifungal. In some embodiments, the subject in need suffers from cancer or a subject having cancer is selected to receive an anti-cancer therapy. In some embodiments, the subject is selected to receive an analgesic.

In some embodiments, the compositions further include a growth factor. Table 1 provides a partial list of growth factors used to accelerate the repair of chronic wounds in humans. Table 2 provides results of double blind, placebo controlled trials of growth factors and chronic wounds.

TABLE 1
Partial list of growth factors used to accelerate
the repair of chronic wounds in humans

| | | Selected | Selected | |
|---|---|---|---|---|
| | Cell or | Target | Stimulatory (S) | |
| | Tissue of | Cells or | or Inhibitory | Clinical |
| Factor | Origin | Tissue | (I) Actions | Trials |
| EGF | macrophages, | epithelium, | S: proliferation | venous |
| | monocytes | endothelial | of keratinocytes, | ulcers |
| | | cells | fibroblasts, and | |
| | | | endothelial cells; | |
| | | | keratinocyte | |
| | | | migration | |
| FGF | macrophages, | endothelium, | S: proliferation | diabetic, |
| | monocytes, | fibroblasts, | of keratinocytes, | pressure, |
| | endothelial | keratinocytes | fibroblasts, and | and |
| | cells | | endothelial cells; | venous |
| | | | chemotaxis, | ulcers |
| | | | ECM | |
| GM- | macrophages, | hematopoietic, | S: IGF-1 | venous |
| CSF | fibroblasts, | inflammatory | production | and |
| | endothelial | cells, | | arterial |
| | cells | neutrophils, | | ulcers |
| | | fibroblasts | | |
| HGH | pituitary | hepatocytes, | S: IGF-1 | venous |
| | gland | bone, | production | ulcers |
| | | fibroblasts | | |
| IL-1 | lymphocytes, | monocytes, | S: monocytes, | pressure |
| | macrophages, | neutrophils, | neutrophils; | ulcers |
| | keratinocytes | fibroblasts, | macrophage | |
| | | keratinocytes | chemotaxis | |
| PDGF | platelets, | fibroblasts, | S: proliferation | diabetic |
| | macrophages, | smooth | of smooth muscle | and |
| | neutrophils, | muscle cells | cells and | pressure |
| | smooth muscle | | fibroblasts; | ulcers |
| | cells | | chemotaxis; | |
| | | | ECM, | |
| | | | contraction | |
| TGF-β | platelets, | fibroblasts, | S: ECM, | venous |
| | bone, most | endothelial | fibroblast, | and |
| | cell types | cells, | activity, | pressure |
| | | keratinocytes, | chemotaxis; | ulcers |
| | | lymphocytes, | I: proliferation | |
| | | monocytes | of keratinocytes | |
| | | | andendothelial | |
| | | | cells | |

EGF = epidermal growth factor; FGF = fibroblast growth factor; GMCSF = granulocyte-macrophage colony-stimulating factor; HGH = human growth hormone; IL-1 = interleukin-1; IGF-1 = insulin growth factor-1; PDGF = platelet-derived growth factor; TGF-β = transforming growth factor-β.

TABLE 2
Double-blind, placebo controlled trials
of growth factors and chronic wounds

| | | Target | | |
|---|---|---|---|---|
| Growth | | Wound | Growth Factor | |
| Factor | Authors | Type (n) | Dose | Results |
| EGF | Falanga et al. | venous (44) | 10 μg/mL twice | N.S. |
| | | | per day | |
| HGH | Rasmussen | venous (37) | 1 IU/cm² per | N.S. |
| | et al. | | week | |
| GM-CSF | da Costa et al. | venous + | 400 μg injected | N.S. |
| | | arterial | once around the | |
| | | (25) | wound | |
| TGF-β2 | Robson et al. | venous (36) | 2.5 μg/cm² three | N.S. |
| | | | times per week | |
| PDGF-BB | Robson et al. | pressure (20) | 1, 10, and 100 | N.S. |
| | | | μg/mL daily | |
| PDGF-BB | Mustoe et al. | pressure (45) | 1 and 3 μg/mL | N.S. |
| | | | daily | |
| PDGF-BB | Steed et al. | diabetic | 2.2 μg/cm² daily | p = 0.01 |
| | | (118) | | |
| PDGF-BB | Wieman et al. | diabetic | 30 and 100 | p = 0.007 |
| | | (382) | μg/gm daily | for 100 |
| | | | | μg dose |
| bFGF | Richard et al. | diabetic (17) | 0.25 to 0.75 | N.S. |
| | | | μg/cm² daily | |
| bFGF | Robson et al. | pressure (50) | 1, 5, and 10 | N.S. |
| | | | μg/cm² | |
| IL-1B | Robson et al. | pressure (25) | 0.01, 0.1, and 1 | N.S. |
| | | | μg/cm² daily | |

EGF = epidermal growth factor; HGH = human growth hormone; GMCSF = granulocyte-macrophage colony-stimulating factor; TGF - β 2 = transforming growth factor-β2; PDGF-BB = platelet-derived growth factor BB; bFGF = basic fibroblast growth factor; IL-1B = interleukin-1B; N.S. = not statistically significant.

VEGF is a signaling protein that promotes the growth of new blood vessels. VEGF forms part of the mechanism that restores the blood supply to cells and tissues when they are deprived of oxygenated blood due to compromised blood circulation.

HGF gene encodes a protein that binds to the hepatocyte growth factor receptor to regulate cell growth, cell motility, and morphogenesis in numerous cell and tissue types. This protein is secreted by mesenchymal cells and acts as a multi-functional cytokine on cells of mainly epithelial origin. This protein also plays a role in angiogenesis, tumorigenesis, and tissue regeneration. Human HGF is expressed as a linear, polypeptide-precursor glycoprotein containing 697 amino acid residues. HGF regulates the chemotaxis of T cells into heart tissue. Binding of HGF by cMet, expressed on T cells, causes the upregulation of cMet, CXCR3, and CCR4 which in turn imbues them with the ability to migrate into heart tissue. HGF has been shown to interact with the protein product of the C-Met oncogene, identified as the HGF receptor (HGFR). Both overexpression of the Met/HGFR receptor protein and autocrine activation of Met/HGFR by simultaneous expression of the hepatocyte growth factor ligand have been implicated in oncogenesis.

KGF is also known as FGF-7 and heparin-binding growth factor-7 (HBGF-7). KGF is a member of the fibroblast growth factor family and has been found to stimulate hair growth. When applied directly to the scalp, KGF binds to KGF receptors on the cell surface, acting as both a growth and survival factor by stimulating epithelial cell proliferation, differentiation, and migration and promoting a number of cell protective mechanisms, thereby stimulating hair growth and increasing the health of the skin. This behavior is especially beneficial to those persons that experience hair loss due to the effects of aging or the side effects of chemotherapy.

Cells that respond to KGF do so because they have receptors on the cell membrane that recognize the growth factor, normally produced by cells near or far from the target cell. The binding of KGF to the receptor initiates a cascade of molecular events that will eventually lead, among other effects, to cell division. KGF has been shown to regulate proliferation and differentiation in epithelial tissues and may regulate the stem cells of the hair follicle.

In some embodiments, the topical formulation further includes, for example, at least one growth factor. In some embodiments, the at least one growth factor is epidermal growth factor (EGF), platelet derived growth factor (PDGF), fibroblast growth factor (FGF), transforming growth factors (TGF-α and TGF-β), nerve growth factor (NGF), erythropoietin (EPO), insulin-like growth factors (IGF-I and IGF-II), interleukin cytokines (IL-1α, IL-1β, IL-2, IL-3, IL-4, IL-5, IL-6, IL-7, IL-8, IL-9, IL-10, IL-11, IL-12, IL-13), interferons (IFN-α, IFN-β, and IFN-γ), tumor necrosis factors (TNFα and TNF-β), colony stimulating factors (GM-CSF and M-CSF). Examples of growth factors (GFs) are described in U.S. Pat. No. 8,518,879 issued Aug. 27, 2013 and U.S. Pat. No. 9,119,974 issued Sep. 1, 2015, both incorporated by reference in their entirety herein.

In some embodiments, the topical formulation further includes one or more bioactive fragmented peptide. In some embodiments, the bioactive fragmented peptide is a collagenase-derived biologically active fragment, a tigerinin-based peptide, or combinations thereof. In some embodiments, the bioactive fragmented peptide is a salamander skin peptide, such as a tylotoin-based peptide. In some embodiments, the fragmented peptide is a frog skin peptide, such as a tigerinin-based peptide.

In some embodiments, the topical formulation can include at least one thickener, at least one humectant, and/or at least one preservative. Thickeners can include, for example, triglycerides, palmitates, myristates, stearates, polyethylene glycol, vegetable-based fatty alcohols, copolymers, cellulose gum, or xanthan gum. Humectants can be used for their moisturizing capabilities. Without being limiting, humectants can include but are not limited to sodium PCA, nanolipid gels, glycerin, alpha-hydroxy acid, butylene glycol, propylene glycol, hexylene glycol, sorbitol, hyaluronic acid, urea, glyceryl triacetate, neoagarobiose, glycerol, xylitol, maltitol, polymeric polyols, polydextrose, quillaia, MP diol, seaweed and algae extracts, and lactic acid.

In some embodiments, the topical formulation further includes at least one preservative. Without being limiting, preservatives can include benzoin resin, jojoba, vitamin E, alcohol, phenoxyethanol, methylparaben, propylparaben, diazolidinyl urea, sorbic acid, and triclosan. In some embodiments, the at least one preservative is benzoin resin, jojoba, vitamin E, alcohol, phenoxyethanol, methylparaben, propylparaben, diazolidinyl urea, sorbic acid, and/or triclosan.

In some embodiments, the formulations further include any one or more of human fibroblast conditioned media, fulvic acid, water, glycerin, polysorbate 20, cellulose gum, tetrahexyldecyl ascorbate, tocopheryl acetate, lactic acid, citrus aurantium bergamia (Bergamot) fruit oil, phenoxyethanol, 1,2-hexanediol, caprylyl glycol, cannabidiol, SD alcohol 40, propylene glycol, menthyl lactate, sodium hyaluronate, squalane, polyglyceryl-6 distearate, cetearyl alcohol, raphanus sativux (radish) seed extract, Jojoba esters, Butyrospermum Parkii (Shea) Oil, Simmondsia Chinensis (Jojoba) Seed Oil, Camellia Sinensis Leaf Extract, Sodium Hyaluronate, Tocopherol, Bisabolol, Dimethicone, Xanthan Gum, Sclerotium Gum, Pullulan, Polyglyceryl-3 Beeswax, Cetyl Alcohol, Terminalia Ferdinandiana Fruit Extract, Alcohol Denat, Saccharide, Isomerate, Citric Acid, Sodium Citrate, Sodium Acrylates Copolymer, Lecithin, Citrus Sinensis (Orange) Oil, Helianthus Annuus (Sunflower) Seed Oil, Citrus Aurantium, Bergamia (Bergamot) Fruit Oil, Pogostemon Cablin Oil, Pelargonium Graveolens Flower Oil, Cananga Odorata Flower Oil, Citrus Limon (Lemon) Peel Oil, Eugenia, Caryophyllus (Clove) Bud Oil, Cymbopogon Martini Oil, Citrus Aurantium Dulcis (Bitter Orange) Peel Oil, Citrus Aurantium Amara (Bitter Orange) Leaf/Twig Oil , Cistus, Ladaniferus Resin, Sandalwood Oil , Rosmarinus Officinalis (Rosemary) Leaf Oil, Ethylhexylglycerin, cocos nucifera (coconut) oil, phospholipids, sorbic acid, sodium benzoate, niacinamide, ethylhexylglycerin, aminopropyl ascorbyl phosphate, xanthan gum, menthyl lactate, carrageenan, sodium hydroxide, or Aloe barbadensis (Aloe) leaf juice.

Without being limiting, the formulation as described herein, can be within a lotion, a cream, a gel, a cosmetic (make-up), sunscreen or a sunblock. Make-up, which can contain the formulation, can include but is not limited to foundation, blush, BB cream, CC cream, foundation primer, primer, lipstick, lip-gloss, eyelash primer, eyeshadow, cream eyeshadow, cream foundation, skin serum, and concealer.

When methods of treating a subject is required, for example, when the subject has inflammation on the scalp, the formulation can be provided in a shampoo, a conditioner, a hairspray, a mousse, a gel, or a hair rinse.

When methods of treating a subject is required, for example, when the subject has inflammation on the nails or surrounding cuticle region, the formulation can be provided in a gel, a lotion, a cream, or a cuticle oil.

When methods of treating a subject is required, for example, when the subject has inflammation nasal passages or surrounding area, the formulation can be provided as a nasal spray or nasal drops.

When methods of treating a subject is required, for example, when the subject has inflammation in the mouth or oral area such the gums lip, inner cheeks or roof of the mouth, the formulation can be provided as a mouth wash, toothpaste, a prophylactic paste, a tooth polish, a dental solution, an oral spray, dental floss, chewing gum, a lozenge, tablet, mouth rinse, or gel/cream within a teeth tray.

When methods of treating a subject are required, for example, when the subject has inflammation in the perineal area, the formulation can be provided as a suppository, cream, gel, ointment, or a lotion.

When methods of treating a subject is required, for example, when the subject has inflammation in the ear or surrounding areas of the ear, the formulation can be provided as medication formulated for the ears such as ear drops.

When methods of treating a subject is required, for example, when the subject has inflammation in the eye or surrounding areas of the eye, the formulation can be provided as medication formulated for the eyes such as eye drops, eye ointments or eye cream. In some embodiments, when the roots of the eyelashes are affected by inflammation, the formulation can be used in an eyelash primer, and the eyelash primer may be administered with a mascara brush or a small brush against the lash line.

When methods of treating a subject are required, for example, when the subject has inflammation of the vaginal area, the formulation can be provided as a cream, gel, ointment, lotion, or vaginal suppository.

A patient suffering from a skin disorder can be treated with an isolated fulvic acid and a cannabinoid, optionally with hyaluronic acid, alone or in combination with other therapies known to treat the disease or condition, or alone or in combination with one or more growth factors and/or one or more bioactive fragmented peptides. As used herein, “therapy” includes but is not limited to a known drug. In addition, the compositions described herein can be combined with a drug associated with an undesirable side effect.

In some embodiments are provided methods of treating a patient diagnosed with a skin disorder or presenting with a skin disorder with a therapeutically effective amount of a composition comprising an isolated fulvic acid and a cannabinoid, optionally with hyaluronic acid, including administering said composition to said patient such that the skin disorder is ameliorated or reduced. Embodiments include methods of treating a patient diagnosed with a skin disorder or presenting with a skin disorder with a therapeutically effective amount of the composition, including administering said composition to said patient such that the symptoms of the skin disorder are reduced or inhibited. In one embodiment, the composition functions by accelerating healing functions, including catalyzing the dynamic processes of wound healing. These processes include: an inflammatory reaction stage consisting of the extravasation of blood constituents with resultant platelet aggregation, blood coagulation, and migration of inflammatory cells to the wound site; a proliferative phase involving the migration and proliferation of keratinocytes, fibroblasts, and endothelial cells, leading to re-epithelialization and granulation tissue formation; and a tissue remodeling phase restoring tissue structural integrity and functional competence.

In some embodiments is provided methods of treating skin disorders with a composition comprising an isolated fulvic acid and a cannabinoid, optionally with hyaluronic acid, as described herein, alone, in combination with growth factors, bioactive fragmented peptides, skin treatments, or in combination with wound or skin therapy by methods known in the art, such as with physical therapy, with pain management treatments, or with other treatments or therapies in the art.

In some embodiments is provided methods of treating skin disorders with a composition comprising an isolated fulvic acid, a cannabidiol, and optionally with hyaluronic acid, the composition is applied to the surface of the skin of a patient suffering from a skin disorder. In some embodiments the composition is a topical formulation that is applied to the surface of the skin for a period of time ranging from about 0.1 to about 120 minutes (min), such as 0.1, 0.5, 1, 2, 3, 4, 5, 6, 7, 8, 9, 10, 11, 12, 13, 14, 15, 16, 17, 18, 19, 20, 25, 30, 35, 40, 45, 60, 90, or 120 min, or an amount within a range defined by any two of the aforementioned values. The topical formulation can penetrate below the surface of the skin to a distance ranging from about 0.5 to about 200 micrometers (μtm), such as 0.5, 1, 5, 10, 15, 20, 30, 40, 50, 60, 70, 80, 90, 100, 110, 120, 130, 140, 150, 160, 170, 180, 190 or 200 μm, or an amount within a range defined by any two of the aforementioned values.

3. Pharmaceutical Formulations

The pharmaceutical compositions of the present disclosure may include an effective amount of an isolated fulvic acid and a cannabinoid, including for example M-007 and cannabidiol, optionally with hyaluronic acid, of the present disclosure in combination with a pharmaceutically acceptable carrier. The compositions may further include one or more growth factors, as described herein, one or more bioactive fragmented peptide, as described herein, or combinations thereof. The compositions may further include other known drugs suitable for the treatment of skin disease or a wound. An effective amount of an isolated fulvic acid and a cannabinoid, optionally with hyaluronic acid, of the present disclosure is an amount that ameliorates the disorder, or which causes the acceleration of the healing process, compared to that which would occur in the absence of the composition comprising an isolated fulvic acid and a cannabinoid, optionally with hyaluronic acid. The effective amount (and the manner of administration) will be determined on an individual basis and will be based on a consideration of the subject (size, age, general health), the severity of the condition being treated, the severity of the symptoms to be treated, the result sought, the specific carrier or pharmaceutical formulation being used, the route of administration, and other factors as would be apparent to those skilled in the art. The effective amount can be determined by one of ordinary skill in the art using techniques as are known in the art. Therapeutically effective amounts of the compounds described herein can be determined using in vitro tests, animal models or other dose-response studies, as are known in the art. The composition comprising the isolated fulvic acid and the cannabinoid, optionally with hyaluronic acid, of the present disclosure can be used alone or in conjunction with other therapies. The therapeutically effective amount may be reduced when the composition is used in conjunction with another therapy.

The pharmaceutical compositions of the disclosure may be prepared, packaged, or sold in formulations suitable for intradermal, intravenous, subcutaneous, oral, rectal, vaginal, parenteral, intraperitoneal, topical, pulmonary, intranasal, buccal, ophthalmic, intrathecal, epidural, or another route of administration. The compounds may be administered by any convenient route, for example by infusion or bolus injection, by absorption through epithelial or mucocutaneous linings (e.g., oral mucosa, rectal, and intestinal mucosa, etc.), and may be administered together with other biologically active agents. Administration can be systemic or local. For example, the pharmaceutical compositions of the disclosure can be administered locally to a tumor via microinfusion. Further, administration may be by a single dose or a series of doses.

The present disclosure thus also provides pharmaceutical compositions suitable for administration to a subject. The carrier can be a liquid, so that the composition is adapted for parenteral administration, or can be solid, i.e., a tablet or pill formulated for oral administration. Further, the carrier can be in the form of a nebulizable liquid or solid so that the composition is adapted for inhalation. When administered parenterally, the composition should be pyrogen free and in an acceptable parenteral carrier. Active compounds can alternatively be formulated or encapsulated in liposomes, using known methods. Other contemplated formulations include projected nanoparticles and immunologically based formulations.

Liposomes are completely closed lipid bilayer membranes that contain entrapped aqueous volume. Liposomes are vesicles that may be unilamellar (single membrane) or multilamellar (onion-like structures characterized by multiple membrane bilayers, each separated from the next by an aqueous layer). The bilayer is composed of two lipid monolayers having a hydrophobic “tail” region and a hydrophilic “head” region. In the membrane bilayer, the hydrophobic (nonpolar) “tails” of the lipid monolayers orient toward the center of the bilayer, whereas the hydrophilic (polar) “heads” orient toward the aqueous phase.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

### Combinations of M-007 and Cannabidiol

This example shows the therapeutic effects of combining M-007 with cannabidiol.

A composition having fulvic acid isolated by providing an aqueous slurry having humified organic matter (HOM), applying the aqueous slurry to high pressure column fractionation to obtain fractionated samples, applying the fractionated samples to molecular sieving, and isolating a fulvic acid, which results in an isolated fulvic acid having an average molecular weight of about 300 Da to 320 Da and a molecular formula of C₁₂H₁₆O₉. The fulvic acid is combined with cannabidiol in a topical formulation, suitable for topical application to a subject. In some embodiments, the compositions were further formulated with hyaluronic acid.

Compositions were prepared and formulated as a face serum, which included HPL human fibroblast conditioned media, M-007 fulvic acid, water, glycerin, polysorbate 20, cellulose gum, tetrahexyldecyl ascorbate, tocopheryl acetate, lactic acid, citrus aurantium bergamia (Bergamot) fruit oil, phenoxyethanol, 1,2-hexanediol, caprylyl glycol, and cannabidiol.

Compositions were prepared and formulated as a hair serum, which included water, HPL human fibroblast conditioned media, M-007 fulvic acid, SD alcohol 40, propylene glycol, polysorbate 20, cellulose gum, tetrahexyldecyl ascorbate, tocopheryl acetate, menthyl lactate, lactic acid, sodium hyaluronate, phenoxyethanol, 1,2-hexanediol, caprylyl glycol, and cannabidiol.

Compositions were prepared and formulated as a moisturizer, which included water, tetrahexyldecyl ascorbate, squalane, polyglyceryl-6 distearate, cetearyl alcohol, fulvic acid, glycerin, raphanus sativux (radish) seed extract, Jojoba esters, Butyrospermum Parkii (Shea) Oil, Simmondsia Chinensis (Jojoba) Seed Oil, Camellia Sinensis Leaf Extract, Sodium Hyaluronate, Tocopherol, Bisabolol, Dimethicone, Xanthan Gum, Sclerotium Gum, Pullulan, Polyglyceryl-3 Beeswax, Cetyl Alcohol, Terminalia Ferdinandiana Fruit Extract, Alcohol Denat, Saccharide, Isomerate, Citric Acid, Sodium Citrate, Sodium Acrylates Copolymer, Lecithin, Citrus Sinensis (Orange) Oil, Helianthus Annuus (Sunflower) Seed Oil, Citrus Aurantium, Bergamia (Bergamot) Fruit Oil, Pogostemon Cablin Oil, Pelargonium Graveolens Flower Oil, Cananga Odorata Flower Oil, Citrus Limon (Lemon) Peel Oil, Eugenia, Caryophyllus (Clove) Bud Oil, Cymbopogon Martini Oil, Citrus Aurantium Dulcis (Bitter Orange) Peel Oil, Citrus Aurantium Amara (Bitter Orange) Leaf/Twig Oil, Cistus, Ladaniferus Resin, Sandalwood Oil, Rosmarinus Officinalis (Rosemary) Leaf Oil, Phenoxyethanol, Ethylhexylglycerin, and cannabidiol.

Compositions were prepared and formulated as a facial booster, which included water, cocos nucifera (coconut) oil, phospholipids, cannabidiol, tocopheryl acetate, sorbic acid, sodium benzoate, fulvic acid, glycerin, niacinamide, phenoxyethanol, ethylhexylglycerin, polysorbate 20, aminopropyl ascorbyl phosphate, xanthan gum, menthyl lactate, carrageenan, sodium hydroxide, and Aloe barbadensis (Aloe) leaf juice.

In any of the prepared formulations, cannabidiol was present in an amount ranging from about 0.1 to 100 mg/mL, such as 0.1, 0.2, 0.3, 0.4, 0.5, 0.6, 0.7, 0.8, 0.9, 1, 2, 3, 4, 5, 6, 7, 8, 9, 10, 15, 20, 25, 30, 40, 50, 60, 70, 80, 90, or 100 mg/mL, using a specific CBD isolate, such as cannabidiol, cannabidiolic acid, cannabidiol monomethylether, cannabidiorcol, cannabidivarin, or cannabidivarinic acid.

### Example 2

### Efficacy of Topical Compositions

This example details the efficacy of the composition obtained in Example 1.

Any of the compositions of Example 1 were applied to skin, hair, or scalp of a subject. The compositions regulates over 150 genes in the skin, including, genes related to wound healing and growth factors, genes connected to skin hydration, genes related to skin barrier integrity, genes related to skin pigmentation, genes related to inflammation and immune response, genes involved in ECM breakdown and barrier integrity, genes relevant to acne, genes connected to anti-aging functions, genes linked to antioxidant and stress responses, genes related to regulating circadian rhythm, and/or genes involved in epidermal barrier formation.

Application of the compositions improves wound regeneration, improves wound healing, improves hair growth, improves sun protection, improve skin appearance, and improves a disorder associated with skin.

### Example 3

### Imaging Studies of Topical Compositions

This example shows the skin penetration effects of a topical formulation, such as any formulations of Example 1.

The topical formulations of Example 1 were applied to skin, and measurements of skin penetration were obtained. The topical formulations included a mixture of proteins and were treated with fluorescein to provide a conjugate. Such proteins include, for example, proteins from growth factor families TGFβ, FGF, EGF, VEGF, and various interleukins and cytokines. Conjugates were titrated in serial dilutions and read on a fluorescence plate reader to estimate dilution into whole product for imaging.

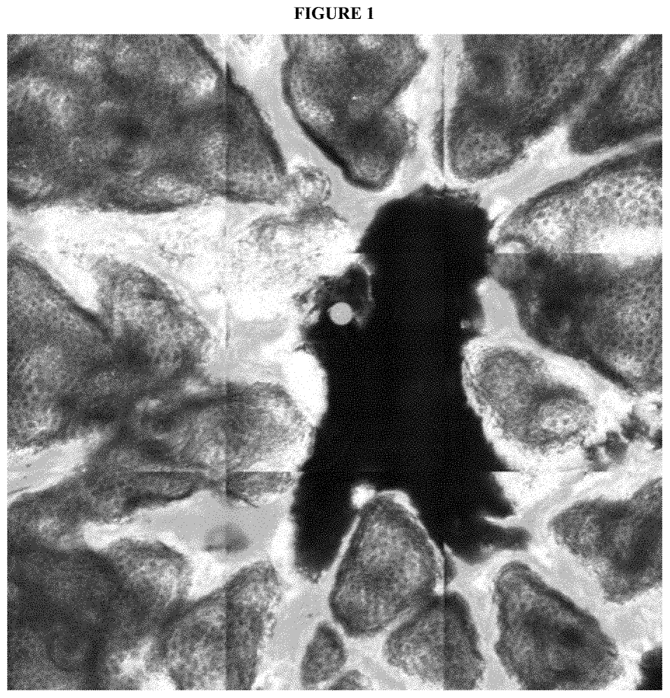

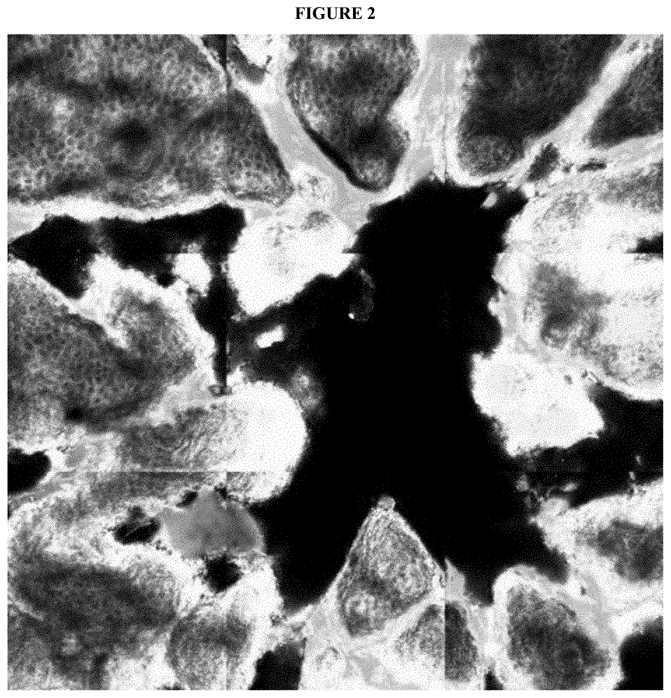

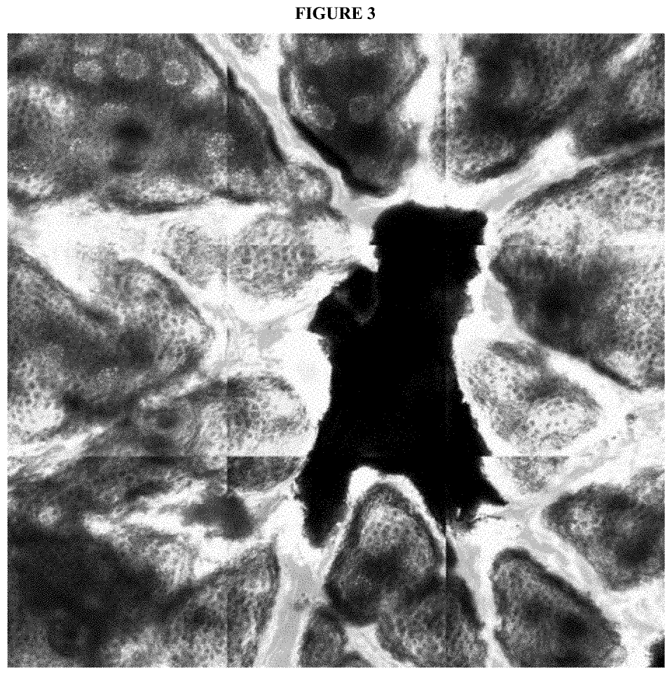

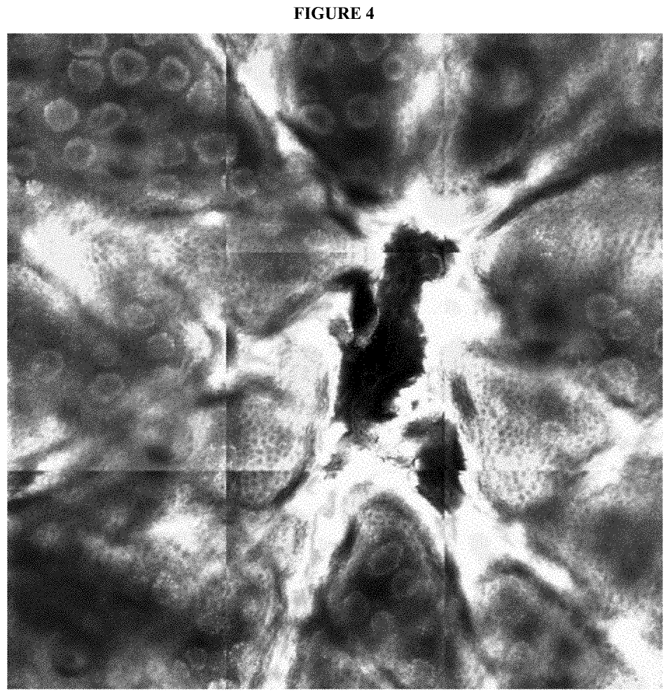

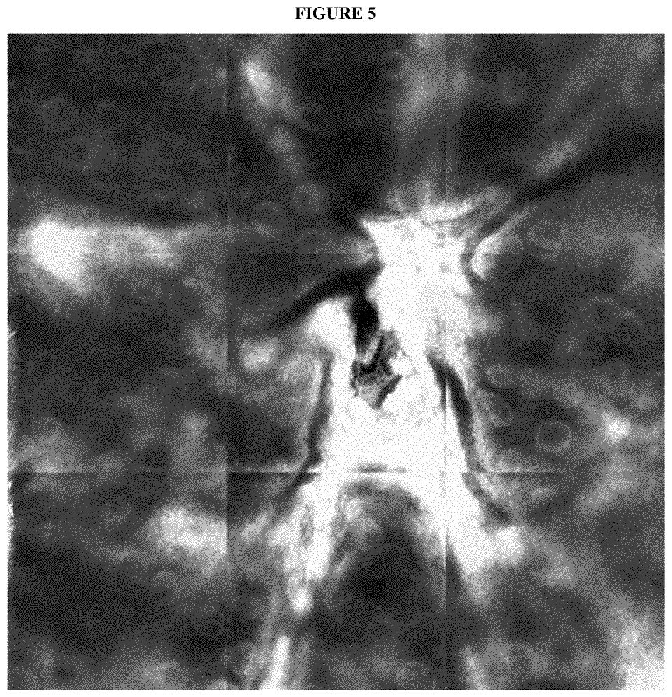

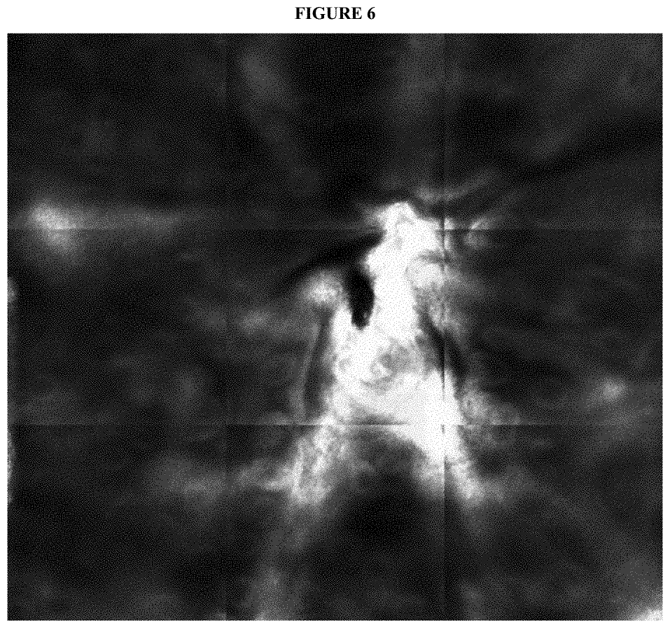

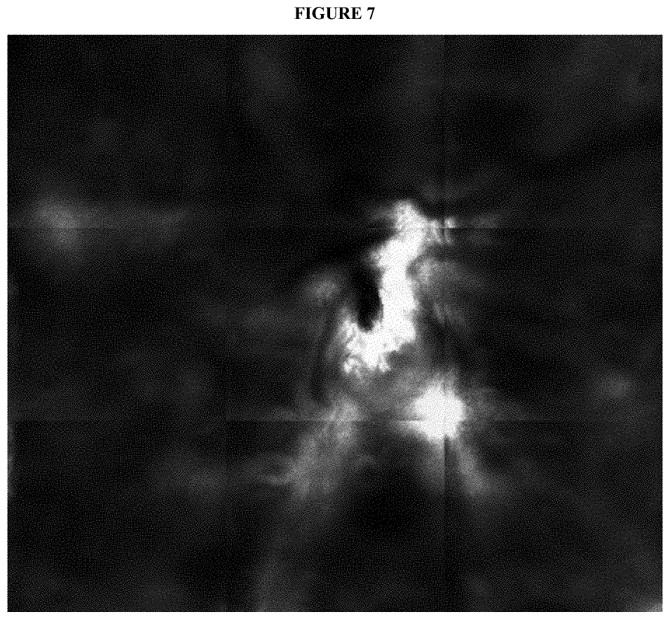

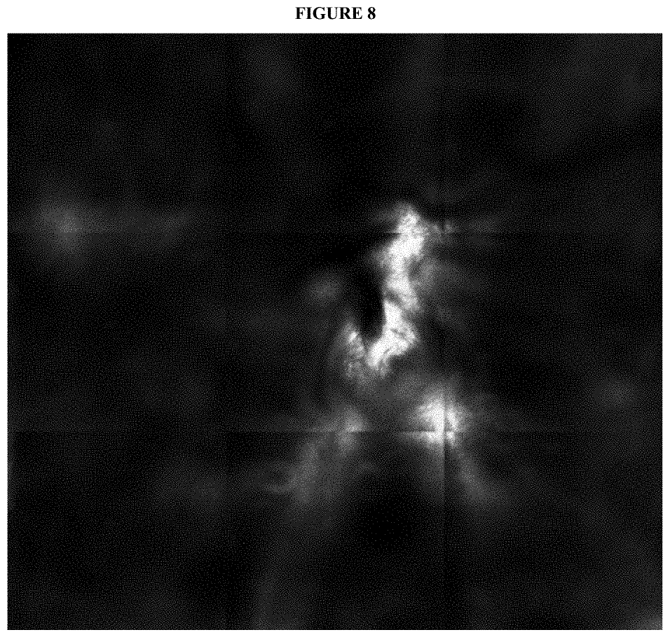

The conjugate was applied to the skin of a subject and allowed to penetrate the subject's skin for 10 minutes. Reflectance and fluorescent confocal microscopy images were collected and merged to display penetration of the conjugate. FIGS. 1-8 display penetration of the combination of M-007 and the mixture of proteins at 0 micrometers (μm), 20 μm, 40 μm, 60 μm, 80 μm, 100 μm, 120 μm and 140 μm, respectively, with green showing the degree of penetration (in grayscale, the degree of penetration is shown in gray), showing that the topical formulations having M-007 result in increased penetration.

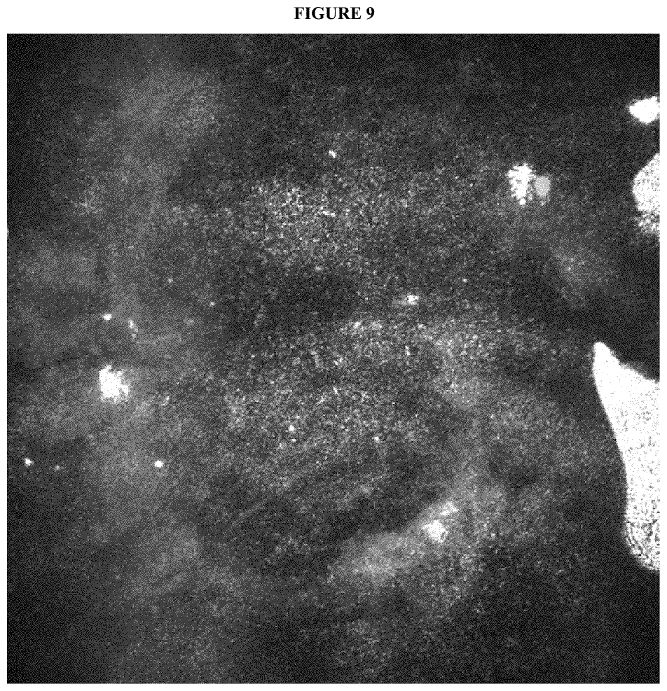

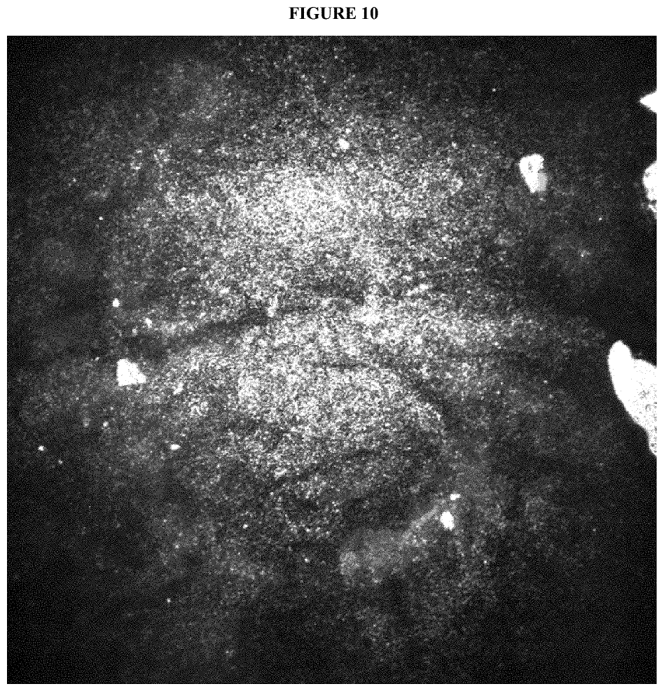

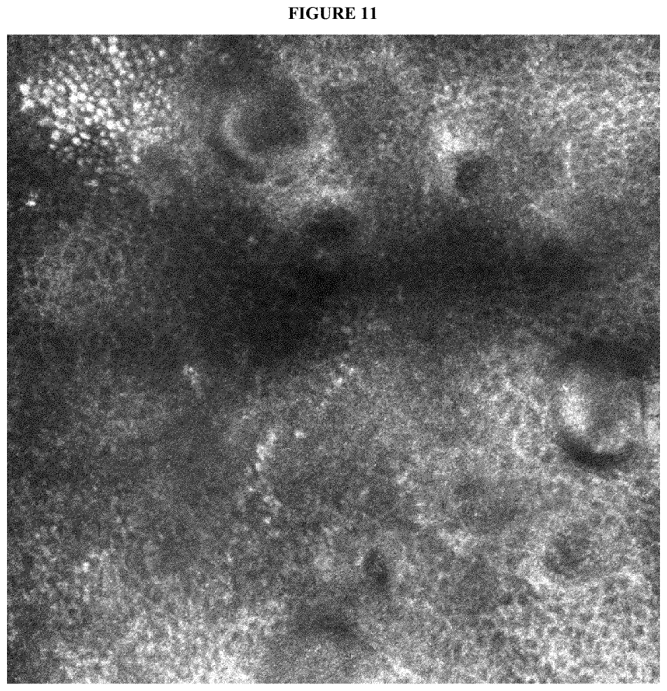

Topical formulations containing no M-007 were prepared by treating the mixture of proteins with fluorescein to provide a conjugate. The conjugate was applied to the skin of a subject and allowed to penetrate the subject's skin for 10 minutes. Reflectance and fluorescent confocal microscopy images were collected and merged. FIGS. 9-11 display no penetration of the mixture of proteins in the absence of M-007 at 30 μm, 60 μm and 100 μm, respectively. These results indicate that in the absence of M-007, the topical formulations do not penetrate the skin, wherein in the presence of M-007, increased skin penetration was ob served.

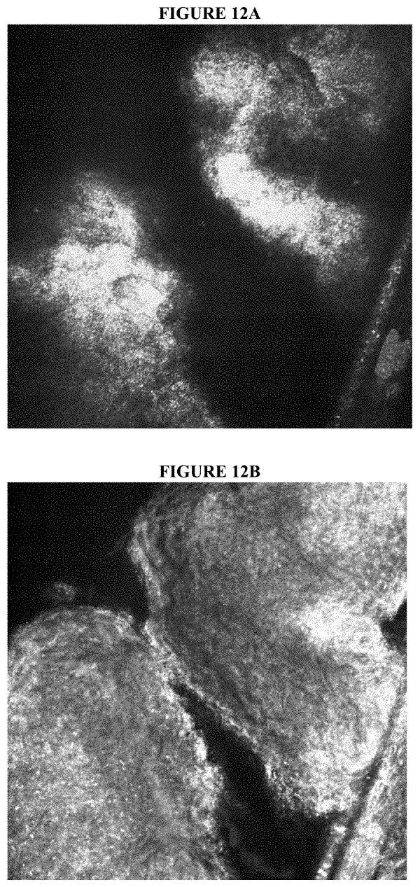

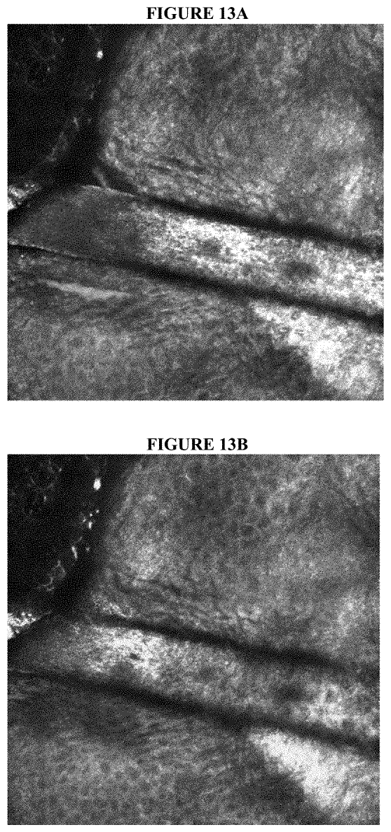

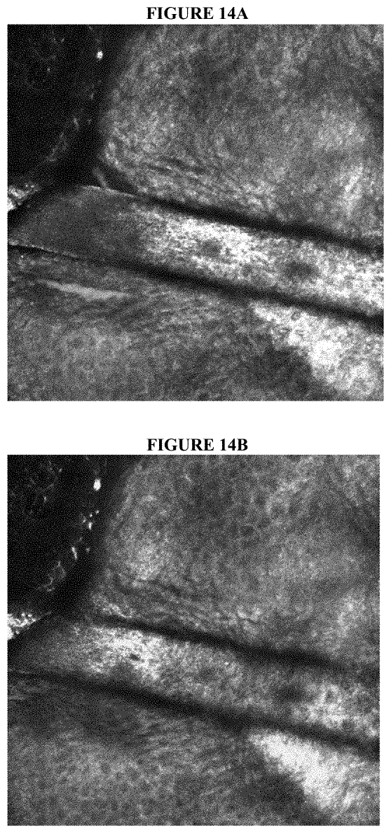

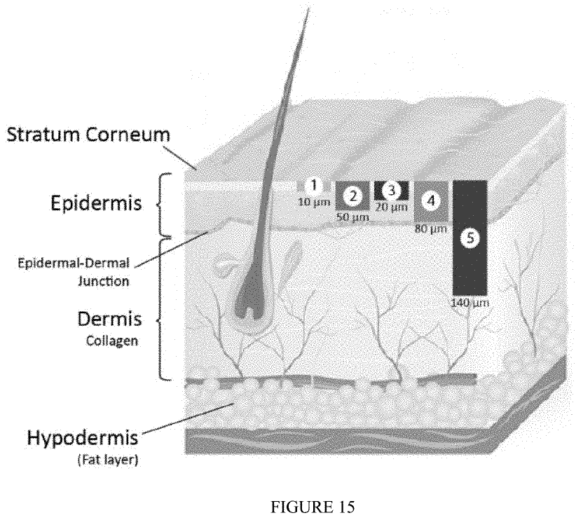

Topical formulations containing no M-007 were prepared by treating three commercially available protein mixtures with fluorescein to provide three conjugates. The commercially available protein mixtures are Serum 2, Serum 3 and Serum 4. These commercially available control serums include various proteins, peptides, and/or growth factors. Each conjugate was applied separately to the skin of a subject and allowed to penetrate the subject's skin for 10 minutes. Reflectance and fluorescent confocal microscopy images were collected and merged. FIGS. 12A and 12B display the extent of penetration for Serum 2 at 10 μm and 50 μm, respectively. FIGS. 13A and 13B display the extent of penetration for Serum 3 at 0 μm and 20 μm, respectively. FIGS. 14A and 14B display the extent of penetration for Serum 4 at 0 μm and 80 μm, respectively. These data demonstrate that commercially available compositions, which lack M-007, were incapable of penetrating the skin. FIG. 15 schematically represents the depth of penetration of the compositions provided herein as compared to commercially available control compositions (1=Serum 1 (topical formulation without M-007, depth of 10 μm); 2=Serum 2 (depth of 50 μm); 3=Serum 3 (depth of 20 μm); 4=Serum 4 (depth of 80 μm); 5=compositions provided herein (depth of 140 μm).

Further experiments demonstrated the skin penetration effects of combining M-007 and fibroblast growth factor (FGF) in a topical formulation. The isolated fulvic acid of Example 1 was combined with FGF in a topical formulation that includes a mixture of proteins and the resultant combination is treated with fluorescein to provide a conjugate. Such proteins include, for example, proteins from growth factor families TGFβ, FGF, EGF, VEGF, and various interleukins and cytokines. The conjugate was applied to the skin of a subject and allowed to penetrate the subject's skin for 10 minutes. Reflectance and fluorescent confocal microscopy images are collected and merged to display penetration of the conjugate.

Reflectance and fluorescent confocal microscopy images were obtained with the VIVASCOPE 1500 confocal imaging system furnished by Caliber I.D.

### Example 4

### Skin Penetration of Topical Compositions

This example details the penetration capabilities of the composition obtained in Example 1.

The compositions of Example 1 are applied to skin of a subject. The compositions, including fulvic acid, cannabidiol, and/or hyaluronic acid is capable of penetration into the skin via topical application more effectively than control compositions that did not include fulvic acid. In particular, the compositions penetrated the skin through the stratum corneum, through the epidermis, and into the dermis to a depth of at least 140 μm. In contrast, control compositions are capable of penetration the skin of the subject to a depth of less than 80 μm.

The depth of penetration results in improved wound regeneration, improves wound healing, improves hair growth, improves sun protection, improve skin appearance, and improves a disorder associated with skin.

These results demonstrate a surprising and unexpected effect of increased skin penetration of the compositions of Example 1, compared to similar compositions that lack isolated fulvic acid fractions. Not only do the compositions of Example 1 result in increased skin penetration compared to samples without isolated fulvic acid fractions, but they also penetrated the skin to a significant depth. The resultant improvement of skin penetration results in improved ability of the compositions provided herein to reach active cell layers below the surface of the skin, resulting in improved treatment, improved skin repair, improved skin quality, and/or decreased aging. More particularly, topical compositions lacking the isolated fulvic acid fractions described herein sit on the surface of the skin, and never reach the active layers below. In contrast, the present compositions disclosed herein, such as the compositions of Example 1, are capable of penetrating to the dermal-epidermal junction and dermis layers of the skin. Penetration to the lower layers of the skin results in improved biological activity, wherein the isolated fulvic acid fractions act as transporters and receivers by both improving penetration of active ingredients in the compositions, and by removing toxins and other particulate matter.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

Unless defined otherwise, technical and scientific terms used herein have the same meaning as commonly understood by one of ordinary skill in the art to which the present disclosure belongs. All patents, applications, published applications and other publications referenced herein are expressly incorporated by reference in their entireties unless stated otherwise. For purposes of the present disclosure, the following terms are defined below.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A composition for treatment of a subject in need of cell regeneration comprising an isolated fulvic acid having an average molecular weight ranging from 80 to 1200 Da, and a cannabinoid.

2. The composition of claim 1, wherein the isolated fulvic acid has an average molecular weight ranging from 80 to 350 Da.

3. The composition of claim 1, wherein the isolated fulvic acid has an average molecular weight ranging from 300 to 320 Da.

4. The composition of claim 1, wherein the isolated fulvic acid has an average molecular weight of about 308.24 Da.

5. The composition of claim 1, wherein the isolated fulvic acid has an average molecular weight of 309 Da.

6. The composition of claim 1, wherein the isolated fulvic acid has an approximate molecular formula of C₁₂H₁₆O₉.

7. The composition of claim 1, wherein the cannabinoid is cannabidiol.

8. The composition of claim 1, wherein the composition is formulated as a topical or injectable composition.

9. The composition of claim 1, wherein the composition further comprises hyaluronic acid.

10. The composition of claim 1, wherein the cannabinoid is present in an amount ranging from about 0.1 to about 100 mg/mL.

11. The composition of claim 1, comprising:
an isolated fulvic acid having an average molecular weight ranging from 300 to 320 Da;
cannabidiol present in an amount of about 2 mg/mL; and
a skin conditioning agent.

12. The composition of claim 11, wherein the skin conditioning agent is human fibroblast conditioned media.

13. The composition of claim 11, further comprising hyaluronic acid, tocopheryl acetate, phenoxyethanol, polysorbate 20, propylene glycol, 1,2-hexanediol, caprylyl glycol, or combinations thereof.

14. The composition of claim 1, wherein the composition is formulated as a wound care formulation, a skin conditioner, a face serum, a hair serum, a moisturizer, or a facial booster.

15. A method for relieving, improving, or causing regression of a wound or skin condition in a subject in need thereof, comprising:
selecting a subject in need thereof; and
topically applying a therapeutically effective amount of a topical composition comprising an isolated fulvic acid and a cannabinoid, wherein the fulvic acid has an average molecular weight of 309 Da and an approximate molecular formula of C₁₂H₁₆O₉, and wherein the cannabinoid is cannabidiol.

16. The method of claim 15, wherein the composition further comprises hyaluronic acid.

17. The method of claim 15, wherein the subject suffers from one or more surgical, accidental, or chronic wound or skin condition.

18. The method of claim 15, wherein the skin condition is rhytide, non-enzymatic glycosylation of the skin, sun damage, smoking damage, fibrosis of the skin, acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, dyshidrosis, acne mechanica, acne medicamentosa, acne miliaris necrotica (acne varioliformis), acne vulgaris, acne with facial edema (solid facial edema), blepharophyma, erythrotelangiectatic rosacea (erythematotelangiectatic rosacea, vascular rosacea), excoriated acne (acne excoriée des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobate, synovitis-acne-pustulosis-hyperostosis-osteomyelitis syndrome (SAPHO syndrome), steroid rosacea, tar acne, skin cancer (carcinoma and melanoma), tropical acne, plaque psoriasis, guttate psoriasis, inverse psoriasis, pustular psoriasis, erythrodermic psoriasis, nail psoriasis, and psoriatic arthritis.

19. The method of claim 15, wherein the topical composition is applied to a surface of the skin of the subject and penetrates below the surface of the skin to a distance ranging from 20 to 200 micrometers (μm).

20. The method of claim 15, wherein the topical composition penetrates below the surface of the skin to a depth of 140 μm.
